(19) United States
(12) Patent Application Publication
(10) Pub. No.: US 2020/0060532 A1
(43) Pub. Date: Feb. 27, 2020

(54) ENDOSCOPE APPARATUS

(71) Applicant: INTHESMART Co., Ltd., Seoul (KR)

(72) Inventors: Uk Kang, Seoul (KR); Ilhyung Shin, Jeju (KR)

(73) Assignee: INTHESMART Co., Ltd., Seoul (KR)

(21) Appl. No.: 16/509,527

(22) Filed: Jul. 12, 2019

(30) Foreign Application Priority Data
Aug. 24, 2018 (KR) 10-2018-0099124

(51) Int. Cl. A61B 1/06; A61B 1/04; G02B 23/26; G02B 23/24

(52) U.S. Cl. CPC A61B 1/0676; A61B 1/04; A61B 1/00011; G02B 23/2461; G02B 23/26

(57) ABSTRACT

An endoscope apparatus includes an image signal transmitting unit configured to perform a pre-emphasis for the image signal according to preset first gain information or second gain information, and generate a first pre-emphasis signal or a second pre-emphasis signal, a signal transmitter configured to include a first transmission line for transmitting the first pre-emphasis signal and a second transmission line for transmitting the second pre-emphasis signal; an image signal receiving unit configured to perform an equalizing operation according to preset second correction information for the second pre-emphasis signal transmitted through the second transmission line.

### CROSS-REFERENCE TO RELATED APPLICATIONS

This application claims priority under 35 U.S.C. § 119 from Korean Application No. 10-2018-0099124 filed on Aug. 24, 2018, the subject matter of which is incorporated herein by reference.

### BACKGROUND OF THE INVENTION

### 1. Field of the Invention

The present invention relates to an endoscope apparatus.

### 2. Description of the Related Art

An endoscope apparatus is inserted into a human organ or an object to acquire an image of a target object, and then transmits the image until it is displayed on a display unit.

What is most important in this process is how to transmit image information to the display unit without loss.

In recent years, the number of pixels of an image pickup device has increased and the resolution of the display unit has been explosively increased. Therefore, it is essential to secure a safe transmission line for stable transmission.

At present, there are no major problems in a high-definition display, but it is essential to improve the stability of transmission lines for high-speed transmission as it is developed to UHD or 4K/8K UHD.

Accordingly, studies are being made on a technology of checking the transmission of image information in real time so as not to stop the medical operation when medical images are cut off and transmitting a stable image through an alternative line when an abnormality occurs in a transmission line.

### SUMMARY OF THE INVENTION

The present invention has been made in view of the above problems, and provides an endoscope apparatus for ensuring stable transmission of an image signal.

In accordance with an aspect of the present invention, an endoscope apparatus includes: a light guide unit configured to guide a light of a light emitting unit into a target object; an image sensing unit configured to sense the light that is arrived after being reflected by the target object and generate an image signal; an image signal transmitting unit configured to perform a pre-emphasis for the image signal according to preset first gain information or second gain information, and generate a first pre-emphasis signal or a second pre-emphasis signal; a signal transmitter configured to include a first transmission line for transmitting the first pre-emphasis signal and a second transmission line for transmitting the second pre-emphasis signal; an image signal receiving unit configured to perform an equalizing operation according to preset second correction information for the second pre-emphasis signal transmitted through the second transmission line, when a post-processing image signal generated by performing the equalizing operation according to preset first correction information with respect to the first pre-emphasis signal is abnormal; and an image signal processing unit configured to process the image signal outputted from the image signal receiving unit and display the processed image signal on a display unit.

The signal transmitter includes a dualized transmission line switching unit configured to switch from the first transmission line to the second transmission line, when the post-processing image signal is abnormal.

The first gain information and the first correction information are used to correct signal distortion according to an impedance of the first transmission line, and the second gain information and the second correction information are used to correct signal distortion according to an impedance of the second transmission line.

The image signal transmitting unit stores a transmission lookup table including the first gain information and the second gain information, and the image signal receiving unit stores a reception lookup table including the first correction information and the second correction information.

The image signal transmitting unit further comprises a monitoring transmission line for transmitting a first hash value generated in a process of being compressed by a hash function, when the image signal transmitting unit performs the pre-emphasis for the image signal compressed by the hash function, and the image signal receiving unit determines whether the post-processing image signal is normal by comparing a second hash value generated according to the hash function after decompressing the post-processing image signal with the first hash value.

A transmission speed of the monitoring transmission line is slower than a transmission speed of the first transmission line and the second transmission line.

The first pre-emphasis signal transmitted through the first transmission line includes redundant information, and the image signal receiving unit checks the redundant information from the post-processing image signal to determine whether the post-processing image signal is normal.

The signal transmitter includes: a monitoring transmission line configured to transmit a switching control signal from the image signal receiving unit, when a check result of the redundant information is abnormal; and a transmission line switching unit configured to switch from the first transmission line to the second transmission line according to the switching control signal.

In accordance with another aspect of the present invention, a method of transmitting a signal by an endoscope apparatus includes: guiding a light of a light emitting unit into a target object through a light guide unit; sensing the light that is arrived after being reflected by the target object by an image sensing unit and generating an image signal; performing a pre-emphasis for the image signal according to preset first gain information and generating a first pre-emphasis signal to transmit the first pre-emphasis signal to a first transmission line; determining whether a post-processing image signal generated by performing an equalizing operation according to preset first correction information with respect to the first pre-emphasis signal is abnormal; performing a pre-emphasis for the image signal according to preset second gain information and transmitting a second pre-emphasis signal through a second transmission line, when the processed image signal is abnormal; performing the equalizing operation according to preset second correction information with respect to the second pre-emphasis signal

The method further includes a dual transmission process for switching from the first transmission line to the second transmission line, when the post-processing image signal is abnormal.

The first gain information and the first correction information are used to correct signal distortion according to an impedance of the first transmission line, and the second gain information and the second correction information are used to correct signal distortion according to an impedance of the second transmission line.

The first gain information and the second gain information are stored in a transmission lookup table, and the first correction information and the second correction information are stored in a reception lookup table including.

The method further includes: transmitting a first hash value generated in a process of being compressed by a hash function through a monitoring transmission line different from the first transmission line and the second transmission line, when the pre-emphasis is performed for the image signal compressed by the hash function, and determining whether the post-processing image signal is normal by comparing a second hash value generated according to the hash function after decompressing the post-processing image signal with the first hash value.

A transmission speed of the monitoring transmission line is slower than a transmission speed of the first transmission line and the second transmission line.

The first pre-emphasis signal transmitted through the first transmission line includes redundant information. The method further includes checking the redundant information from the post-processing image signal and determining whether the post-processing image signal is normal.

The method further includes: transmitting a switching control signal from the image signal receiving unit, when a check result of the redundant information is abnormal; and a dual transmission process of switching from the first transmission line to the second transmission line according to the switching control signal.

### BRIEF DESCRIPTION OF THE DRAWINGS

The above and other objects, features and advantages of the present invention will be more apparent from the following detailed description in conjunction with the accompanying drawings, in which:

FIG. 1 illustrates an endoscope apparatus according to an embodiment of the present invention;

FIG. 2 illustrates a signal transmitting unit of an endoscope apparatus according to an embodiment of the present invention;

FIG. 3 and FIG. 4 are diagrams for explaining the operation of an endoscope apparatus according to an embodiment of the present invention; and

FIG. 5 is a flowchart of a signal transmission method of an endoscope apparatus according to an embodiment of the present invention.

### DETAILED DESCRIPTION OF THE PREFERRED EMBODIMENTS

Exemplary embodiments of the present invention are described with reference to the accompanying drawings in detail. The same reference numbers are used throughout the drawings to refer to the same or like parts. Detailed descriptions of well-known functions and structures incorporated herein may be omitted to avoid obscuring the subject matter of the present invention. The terminology used herein is for the purpose of describing particular example embodiments only and is not intended to be limiting of the present inventive concept. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

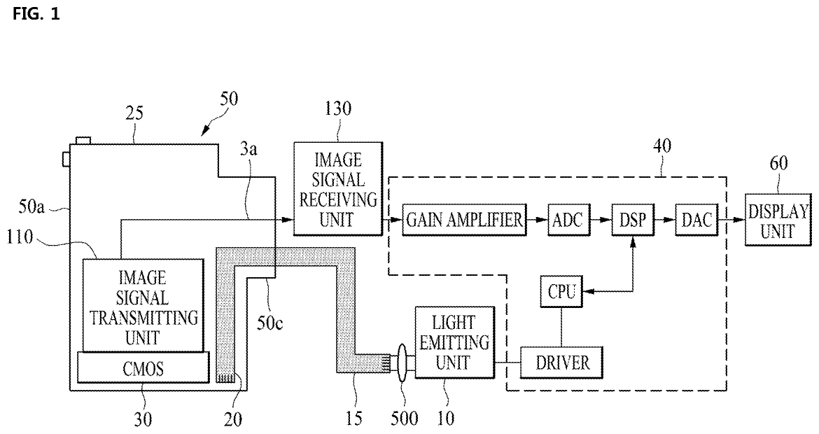

FIG. 1 illustrates an endoscope apparatus according to an embodiment of the present invention. As shown in FIG. 1, the endoscope apparatus according to an embodiment of the present invention includes an endoscope 50 for observing light of a specific wavelength, an image signal processing unit 40 for driving the endoscope 50 and processing an image signal captured by the endoscope 50, and a display unit for displaying an image of the captured target object.

The endoscope 50 includes an image sensing unit 30 configured to sense a reached light that is reflected from a target object and generate an image signal, a flexible or rigid insertion unit 50a configured to be inserted into the target object to which light hardly reaches, and a handle 25 configured to be provided to the insertion unit 50a, and a universal code unit 50c configured to extend from the side of the handle 25. The endoscope 50 is electrically connected the image signal processing unit 40 through the universal code unit 50c.

The image sensing unit 30 may include an image sensing device such as a Charge Coupled Device (CCD) or a Commentary Metal-Oxide Semiconductor (CMOS), but the image sensing device is not limited thereto. In addition, a main body unit of the endoscope 50 may be mainly constituted of the insertion unit 50a and the handle 25. An image signal transmitting unit 110 may be provided in the main body unit. The image signal transmitting unit 110 may generate first and second pre-emphasis signals, which will be described in detail later.

The first and second pre-emphasis signals are inputted to an image signal receiving unit 130 through a signal transmitter 3a. The signal transmitter 3a may include a first transmission line TL1 and a second transmission line TL2 formed of a micro coaxial cable. However, the first transmission line TL1 and the second transmission line TL2 are not limited thereto and may be formed of other types of cables. The image signal processing unit 40 processes the image signal outputted from the image signal receiving unit 130 and enables the display unit 60 to display the processed image signal.

The image signal receiving unit 130 and the image signal processing unit 40 will be described later in detail.

A light guide unit 20 guides light of a light emitting unit 10 into the target object. The light guide unit 20 may be connected to the image signal processing unit 40 through the universal code unit 50c from the insertion unit 50a. The light guide unit 20 may guide the light emitted from the light emitting unit 10 to be outputted to the end of the insertion unit 50a.

The image signal processing unit 40 may include a gain amplifier, an analog-digital converter (ADC), a digital signal processor (DSP), a digital-analog converter (DAC), and a controller CPU. The gain amplifier may amplify the image signal outputted from the image signal receiving unit 130 according to an appropriate gain. The analog-to-digital converter (ADC) may convert the amplified image signal into a digital signal. The digital signal processor (DSP) may perform image processing for an image signal in the form of a digital signal. The digital-to-analog converter (DAC) converts an image signal which has image processed into an analog signal.

The controller (CPU) controls the overall operation of the endoscope apparatus according to the embodiment of the present invention, and may control the image processing process.

The light emitting unit 10 may be driven by being supplied with power by a driver, and the driver may be controlled by the controller (CPU). In this case, the light emitting unit 10 may include an LED, but is not limited thereto.

In FIG. 1, an optical unit 500 is provided between the light emitting unit 10 and an optical fiber 15, but the light of the light emitting unit 10 may be incident on the optical fiber 15 without the optical unit 500.

FIG. 1 is only an example of the endoscope apparatus according to the embodiment of the present invention, and is not limited thereto, and the configuration may be changed as necessary.

Next, the image signal transmitting unit 110, the signal transmitter 3a, and the image signal receiving unit 130 will be described in detail with reference to the drawings.

The image signal transmitting unit 110 pre-emphasizes an image signal according to preset first gain information or second gain information to generate a first or a second pre-emphasis signal.

A first pre-emphasis signal is a result of performing pre-emphasis for the image signal according to the first gain information, and the second pre-emphasis signal is a result of performing pre-emphasis for the image signal according to the second gain information

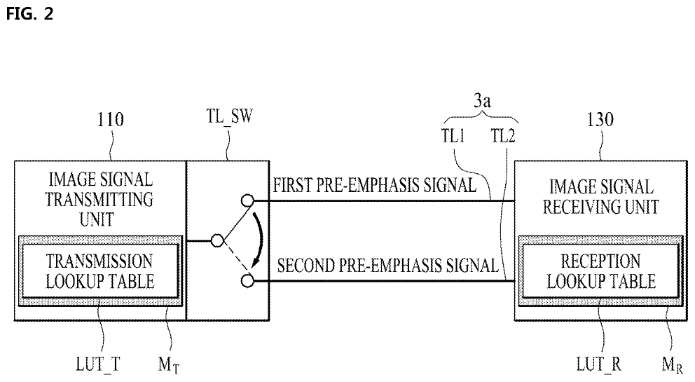

The signal transmitter 3a includes a first transmission line TL1 for transmitting the first pre-emphasis signal and a second transmission line TL2 for transmitting a second pre-emphasis signal. In FIG. 2, the first transmission line TL1 and the second transmission line TL2 are shown, but the present invention is not limited thereto. The signal transmitter 3a may include at least one first transmission line TL1, and at least one second transmission line TL2.

At this time, the signal transmitter 3a may include a transmission line switching unit TL_SW for switching from the first transmission line TL1 to the second transmission line TL2. The signal transmitter 3a is not limited to the configuration of FIG. 2, and various configurations that can switch from the first transmission line TL1 to the second transmission line TL2 may be applied.

The order of switching from the first transmission line TL1 to any second transmission line TL2 among the at least one second transmission line TL2 may be performed according to a preset protocol.

When a post-processing image signal generated by performing equalizing operation according to first correction information with respect to the first pre-emphasis signal is abnormal, the image signal receiving unit 130 performs an equalizing operation according to preset second correction information with respect to the second pre-emphasis signal transmitted through the second transmission line TL2.

The endoscope apparatus is handled by an operator such as a doctor. Since an image of the inside of a human body or an object should be generated, the first transmission line TL1 may be bent or twisted frequently. Accordingly, the first transmission line TL1 may be easily aged.

If the first transmission line TL1 is aged, the impedance of the first transmission line TL1 may be changed. If the impedance of the first transmission line TL1 is changed, a transmission error may occur when a signal is transmitted through the first transmission line TL1.

If such a transmission error occurs, that is, an equalizing operation may be performed according to the first correction information with respect to the first pre-emphasis signal transmitted through the first transmission line TL1, and accordingly, the post-processing image signal may be generated. If such a generated post-processing image signal is abnormal, it is switched from the first transmission line TL1 to the second transmission line TL2 of the endoscope apparatus according to the embodiment of the present invention, thereby performing a stable transmission of the image signal through the dual transmission.

In other words, the image signal generated from the image sensing unit 30 may be pre-emphasized according to first gain information and may be transmitted to the first transmission line TL1. When a transmission error occurs due to the first transmission line TL1, it is switched from the first transmission line TL1 to the second transmission line TL2 so that the image signal of the image sensing unit 30 can pre-emphasized according to second gain information and transmitted to the second transmission line TL2.

The impedance generated by the first transmission line TL1 or the second transmission line TL2 may be calculated by the following Equations 1 and 2. Assuming that the image signal is A sin ω_{d}t, the image signal may be expressed by the following Equation 1. The mathematical expression of the image signal is not limited to A sin ω_{d}t, but may be expressed in various forms having frequency ω_{d} in some cases.

$$L \frac{{d}^{2} q}{{dt}^{2}} + R \frac{dq}{dt} + \frac{1}{C} q = A sin {ω}_{d} t \qquad [ Equaton 1 ]$$

Here, L is the inductance of the transmission line of the first transmission line TL1 or the second transmission line TL2, R is the resistance of the transmission line of the first transmission line TL1 or the second transmission line TL2, and C is the capacitance of the system.

In addition, A is the intensity of the image signal, and q is the electric charge. When the electric charge is differentiated with respect to time, it is changed into current value so that dt/dq is the electric current i.

In this case, the impedance Z of the first transmission line TL1 or the second transmission line TL2 may be expressed by the following Equation 2.

Z=\sqrt{R²+(ωL−1/ω_{d}C)²} [Equation 1]

As described above, as the first transmission line TL1 is aged, the inductance, resistance, and capacitance of the first transmission line TL1 are changed. The first gain information and the first correction information for pre-emphasis and equalizing correspond to the impedance of the first transmission line TL1 before the change. Thus, the signal transmission through the first transmission line TL1 may not be normally performed.

In order to cope with the impedance change of the first transmission line TL1, the gain information and the correction information of the pre-emphasis and the equalizing need to be changed according to the change of the impedance of the first transmission line TL1.

When the pre-emphasis is performed in an active manner differently from the endoscope apparatus according to the embodiment of the present invention, the gain value of the pre-emphasis must be changed whenever the impedance of the first transmission line TL1 is changed.

At this time, if the intensity of the image signal with respect to the impedance change of the transmission line is not appropriately changed as the control of the pre-emphasis is not properly performed, the transmission of the image signal may not be normally performed.

If the transmission of the image signal is not performed smoothly, the doctor may not recognize the internal condition of the patient's body. Thus, a fatal risk, such as a case where surgery or medical procedure should be stopped, may occur.

That is, since the endoscope apparatus is directly connected to the life of the patient, the stable operation of the endoscope apparatus is more important than the stability of other communication apparatuses, and accordingly, the pre-emphasis and equalizing scheme of the active type may not be suitable for the endoscope apparatus.

In order to prevent this, the endoscope apparatus according to the embodiment of the present invention may perform passive type pre-emphasis and equalizing instead of active type pre-emphasis and equalizing.

That is, when the first transmission line TL1 cannot normally transmit an image signal, the endoscope apparatus according to the embodiment of the present invention configure the dualization of the transmission line so that the first transmission line TL1 can be replaced with the second transmission line TL2

Since the impedance of the first transmission line TL1 and the second transmission line TL2 are different from each other due to switching from the first transmission line TL1 to the second transmission line TL2, pre-emphasis and equalizing for the transmission of the image signal of the image sensing unit 30 must be done differently.

Accordingly, the endoscope apparatus according to the embodiment of the present invention performs passive type pre-emphasis and equalizing operations. To this end, the first gain information and the first correction information of the first transmission line TL1 are previously set, and the second gain information and the second correction information of the second transmission line TL2 are previously set.

In this case, the first gain information and the second gain information are for pre-emphasis that changes the intensity of the image signal, and the first correction information and the second correction information are for equalizing that compensates the signal distortion during transmission and adjusts the discrimination ratio of signal.

The discrimination ratio of signal may be a ratio that filters or passes a specific frequency component of a signal transmitted through the first transmission line TL1 or the second transmission line TL2.

The impedance for the first transmission line TL1 and the second transmission line TL2 may be measured in the manufacturing process of the endoscope apparatus according to the embodiment of the present invention. At this time, the first gain information and the first correction information may be previously set through an experiment to determine whether the transmission of the signal is most stably accomplished along the first transmission line TL1 having the measured impedance. The second gain information and the second correction information are also may be previously set through an experiment to determine whether the transmission of the signal is most stably accomplished through the second transmission line TL2 having the measured impedance.

That is, the first gain information and the first correction information may be used to correct the signal distortion due to the impedance of the first transmission line TL1, and the second gain information and the second correction information may be used to correct the signal distortion due to the impedance of the second transmission line. At this time, the image signal transmitting unit 110 may store a transmission lookup table LUT_T containing the first gain information and the second gain information, and the image signal receiving unit 130 may store a reception lookup table LUT_R containing the first correction information and the second correction information.

As described above, the signal transmitter 3a may include at least one first transmission line TL1 and at least one second transmission line TL2, and the transmission lookup table LUT_T may include the first gain information of each transmission line TL1, and the second gain information of each of the second transmission line TL2. In addition, the transmission lookup table LUT_T may include impedance information of each of the first transmission line TL1 and the second transmission line TL2.

Similarly, the reception lookup table LUT_R may include first correction information of each first transmission line TL1 and second correction information of each second transmission line TL2. In addition, the reception lookup table LUT_R may also include impedance information of each of the first transmission line TL1 and the second transmission line TL2.

The image signal transmitting unit 110 and the image signal receiving unit 130 may include memory MT, MR for storing the transmission lookup table LUT_T and the reception lookup table LUT_R.

The memory MT, MR may be nonvolatile memory such as a flash memory that can be written and read. Accordingly, the transmission lookup table LUT_T and the reception lookup table LUT_R can be updated.

For example, due to aging, the signal transmitter 3a may be replaced, and an update on the transmission lookup table LUT_T and the reception lookup table LUT_R corresponding to the impedance of the replaced signal transmitter 3a.

The durability of the endoscope apparatus may be reduced due to bending or twisting of the signal transmitter 3a. Accordingly, there is a high demand for replacement of the signal transmitter 3a for maintenance and repair of the endoscope apparatus.

when the signal transmitter 3a is replaced, the impedance of the signal transmitter 3a is also changed. Thus, in case of an active scheme, the first gain information, the second gain information, the first correction information, and the second correction information of all the first transmission line TL1 and second transmission line TL2 that constitute the signal transmitter 3a should be measured after the replacement of the signal transmitter 3a. Thus, the operation of the endoscope apparatus may become unstable.

On the other hand, since the endoscope apparatus according to the embodiment of the present invention uses a passive scheme, the transmission lookup table LUT_T and the reception lookup table LUT_R optimized for the impedance of the signal transmitter 3a can be updated with replacement of the signal transmitter 3a, thereby achieving more stable operation.

Meanwhile, as described above, the signal transmitter 3a may include a transmission line switching unit TL_SW. When a post-processing image signal which is generated by performing equalizing operation according to the preset first correction information with respect to the first pre-emphasis signal is abnormal, the transmission line switching unit TL_SW may configure a transmission dualization to switch from the first transmission line TL1 to the second transmission line TL2.

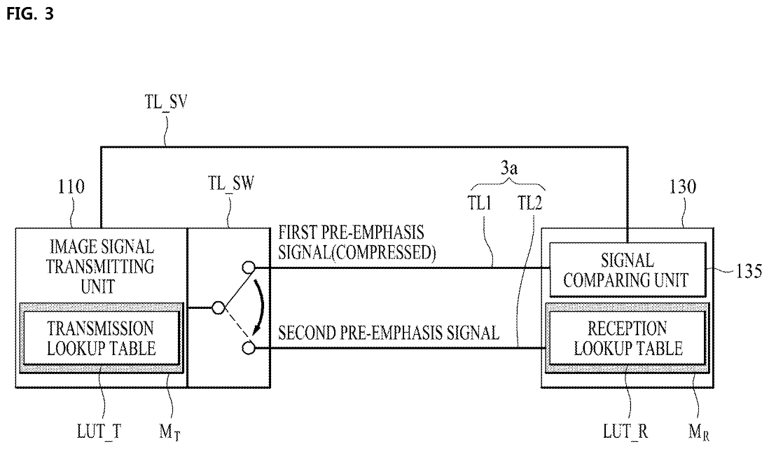

As shown in FIG. 3, the signal transmitter 3a may further include a monitoring transmission line TL_SV. The image signal transmitting unit 110 may compress the image signal by a hash function and pre-emphasize the compressed image signal. In this case, the monitoring transmission line TL_SV may transmit a first hash value generated in the process of being compressed by the hash function.

The image signal receiving unit 130 may generate a second hash value according to a hash function after decompressing the post-processing image signal that has achieved equalizing, and compare the second hash value with the first hash value, thereby determining whether the post-processing image signal is normal.

The image signal receiving unit 130 may include a signal comparing unit 135 for comparing the first hash value and the second hash value. If the signal is normally transmitted, the first and second hash values generated by the image signal transmitting unit 110 and the image signal receiving unit 130 may coincide with each other. If the transmission of the signal is abnormal, the first hash value and the second hash value may coincide with each other.

If the first hash value and the second hash value are not coincide with each other, the signal transmission process is not normally performed. Thus, the transmission line switching unit TL_SW may switch from the first transmission line TL1 to the second transmission line TL2. At this time, the transmission speed of the monitoring transmission line TL_SV may be slower than the transmission speed of the first transmission line TL1 and the second transmission line TL2. That is, the data amount of the hash value generated in the compression process by the hash function may be very smaller than the first pre-emphasis signal and the second pre-emphasis signal transmitted through the first transmission line TL1 and the second transmission line TL2.

Accordingly, even if the first hash value is transmitted to the signal comparing unit 135 at a relatively low speed by the monitoring transmission line TL_SV, it is possible to determine whether the signal transmission is normal by comparing the first hash value with the second hash value.

The transmission line switching unit TL_SW may be controlled by the image signal receiving unit 130, but the present invention is not limited thereto and may be implemented by the CPU of FIG. 1.

FIG. 3 illustrates that the signal comparing unit 135 determines the transmission error of the post-processing image signal of the first pre-emphasis signal. In addition, the normal state of the second pre-emphasis signal may also be determined by comparing the hash value of the post-processing image signal of the second pre-emphasis signal transmitted through the second transmission line TL2.

meanwhile, unlike FIG. 3, it is possible to determine the normal state of the signal transmission through the first transmission line TL1 by a Cyclical Redundancy Check (CRC) code scheme.

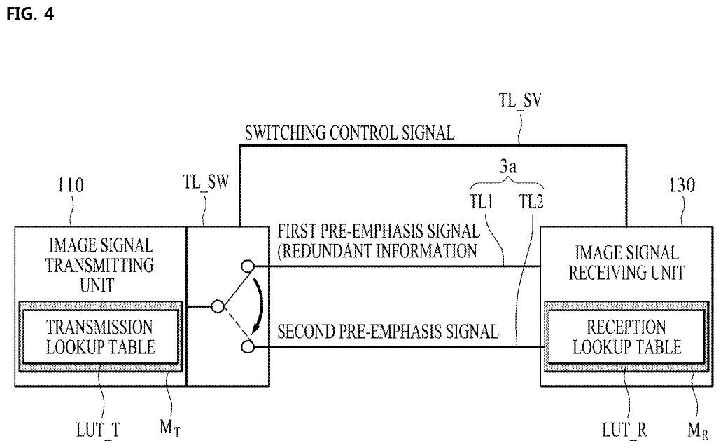

As shown in FIG. 4, the first pre-emphasis signal transmitted through the first transmission line TL1 may include redundant information. The image signal receiving unit 130 may check the redundant information from the post-processing image signal to determine whether the post-processing image signal is normal.

To this end, the image signal transmitting unit 110 may generate redundant information through a generation function, and perform a pre-emphasis operation for the image signal including the redundant information through the first gain information, thereby transmitting the first pre-emphasis signal to the first transmission line TL1. The image signal receiving unit 130 may perform an equalizing operation through the first correction information to generate a post-processing image signal, and check the redundant information included in the post-processing image signal through a check function to determine whether the transmission of the transmitted signal is error.

When the check result of the redundant information is abnormal, the signal transmitter 3a may include a monitoring transmission line TL_SV for transmitting a switching control signal from the image signal receiving unit 130, and a transmission line switching unit TL_SW for switching from the first transmission line TL1 to the second transmission line TL2 according to a switching control signal.

Accordingly, even if the signal transmission through the first transmission line TL1 is not normally performed, the image signal is transmitted through the second transmission line TL2, thereby achieving a stable operation of the endoscope apparatus.

Next, a signal transmission method of an endoscope apparatus according to an embodiment of the present invention will be described with reference to the drawings.

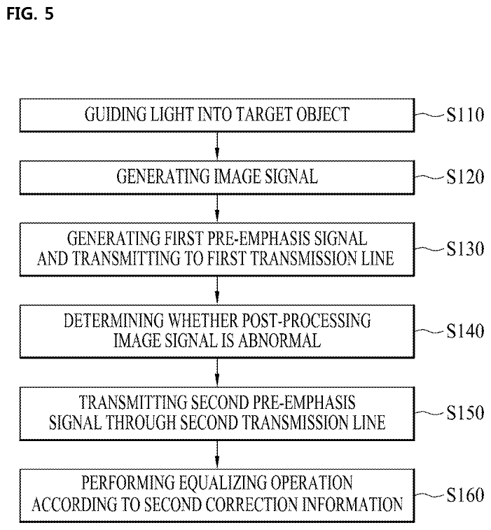

FIG. 5 is a flowchart of a signal transmission method of an endoscope apparatus according to an embodiment of the present invention. The light from the light emitting unit 10 is guided into the target object through the light guide unit 20 (S110).

The image sensing unit 30 senses the light that is arrived after being reflected by the target object, and generates an image signal (S120).

A pre-emphasis is performed for the image signal according to preset first gain information so that a first pre-emphasis signal is generated and transmitted to the first transmission line TL1 (S130).

An equalizing operation is performed according to the preset first correction information for the first pre-emphasis signal to determine whether the generated post-processing image signal is abnormal (S140).

If the post-processing image signal is abnormal, the image signal is pre-emphasized according to the preset second gain information, and the second pre-emphasis signal is transmitted through the second transmission line TL2 (S150).

An equalizing operation is performed according to the set second correction information with respect to the second pre-emphasis signal (S160).

At this time, the first and second pre-emphasis signals may be generated simultaneously, but the second pre-emphasis signal may be generated after the first pre-emphasis signal is generated.

If the post-processing image signal is abnormal, it is possible to switch from the first transmission line TL1 to the second transmission line TL2.

The first gain information and the first correction information may be used to correct the signal distortion caused by the impedance of the first transmission line, and the second gain information and the second correction information may be used to correct the signal distortion caused by the impedance of the second transmission line.

The first gain information and the second gain information are stored in the transmission lookup table LUT_T, and the first correction information and the second correction information may be stored in the reception lookup table LUT_R.

When pre-emphasis is performed on the image signal compressed by the hash function, the first hash value generated in the process of being compressed by the hash function is transmitted through the monitoring transmission line TL_SV different from the first transmission line TL1 and the second transmission line TL2, and it is possible to determine the normal state of the post-processing image signal is by comparing the second hash value generated according to the hash function after decompressing the post-processing image signal with the first hash value.

The transmission speed of the monitoring transmission line TL_SV may be slower than the transmission speed of the first transmission line TL1 and the second transmission line TL2.

The first pre-emphasis signal transmitted through the first transmission line TL1 includes redundant information, and may check the redundant information from the post-processing image signal to determine whether the post-processing image signal is normal.

When the check result of the redundancy information is abnormal, a switching control signal is transmitted through the monitoring transmission line TL_SV which is different from the first transmission line TL1 and the second transmission line TL2, and it is possible to switch from the first transmission line TL1 to the second transmission line TL2 according to the switching control signal.

Although the above description has been made through the endoscope apparatus according to the embodiment of the present invention, it is also possible to implement the image transmission method of the endoscope apparatus according to the embodiment of the present invention through an endoscope apparatus having a different structure from the above described endoscope apparatus.

The endoscope apparatus according to the embodiment of the present invention determines the normal state of the transmitted image signal, switches the transmission line when the image signal is abnormal, performs pre-emphasis and equalizing for impedance matching of the switched transmission signal, thereby maintaining a stable transmission of the image signal.

Although the exemplary embodiments of the present invention have been disclosed for illustrative purposes, those skilled in the art will appreciate that various modifications, additions and substitutions are possible, without departing from the scope and spirit of the invention as disclosed in the accompanying claims. Accordingly, the scope of the present invention is not construed as being limited to the described embodiments but is defined by the appended claims as well as equivalents thereto.

What is claimed is:

1. An endoscope apparatus comprising:
a light guide unit configured to guide a light of a light emitting unit into a target object;
an image sensing unit configured to sense the light that is arrived after being reflected by the target object and generate an image signal;
an image signal transmitting unit configured to perform a pre-emphasis for the image signal according to preset first gain information or second gain information, and generate a first pre-emphasis signal or a second pre-emphasis signal;
a signal transmitter configured to include a first transmission line for transmitting the first pre-emphasis signal and a second transmission line for transmitting the second pre-emphasis signal;
an image signal receiving unit configured to perform an equalizing operation according to preset second correction information for the second pre-emphasis signal transmitted through the second transmission line, when a post-processing image signal generated by performing the equalizing operation according to preset first correction information with respect to the first pre-emphasis signal is abnormal; and
an image signal processing unit configured to process the image signal outputted from the image signal receiving unit and display the processed image signal on a display unit.

2. The endoscope apparatus of claim 1, wherein the signal transmitter comprises a dualized transmission line switching unit configured to switch from the first transmission line to the second transmission line, when the post-processing image signal is abnormal.

3. The endoscope apparatus of claim 1, wherein the first gain information and the first correction information are used to correct signal distortion according to an impedance of the first transmission line,
wherein the second gain information and the second correction information are used to correct signal distortion according to an impedance of the second transmission line.

4. The endoscope apparatus of claim 1, wherein the image signal transmitting unit stores a transmission lookup table including the first gain information and the second gain information,
wherein the image signal receiving unit stores a reception lookup table including the first correction information and the second correction information.

5. The endoscope apparatus of claim 1, wherein the image signal transmitting unit further comprises a monitoring transmission line for transmitting a first hash value generated in a process of being compressed by a hash function, when the image signal transmitting unit performs the pre-emphasis for the image signal compressed by the hash function, and
wherein the image signal receiving unit determines whether the post-processing image signal is normal by comparing a second hash value generated according to the hash function after decompressing the post-processing image signal with the first hash value.

6. The endoscope apparatus of claim 5, wherein a transmission speed of the monitoring transmission line is slower than a transmission speed of the first transmission line and the second transmission line.

7. The endoscope apparatus of claim 1, wherein the first pre-emphasis signal transmitted through the first transmission line includes redundant information,
wherein the image signal receiving unit checks the redundant information from the post-processing image signal to determine whether the post-processing image signal is normal.

8. The endoscope apparatus of claim 7, wherein the signal transmitter comprises:
a monitoring transmission line configured to transmit a switching control signal from the image signal receiving unit, when a check result of the redundant information is abnormal; and
a transmission line switching unit configured to switch from the first transmission line to the second transmission line according to the switching control signal.

9. A method of transmitting a signal by an endoscope apparatus, the method comprising:
guiding a light of a light emitting unit into a target object through a light guide unit;
sensing the light that is arrived after being reflected by the target object by an image sensing unit and generating an image signal;
performing a pre-emphasis for the image signal according to preset first gain information and generating a first pre-emphasis signal to transmit the first pre-emphasis signal to a first transmission line;
determining whether a post-processing image signal generated by performing an equalizing operation according to preset first correction information with respect to the first pre-emphasis signal is abnormal;
performing a pre-emphasis for the image signal according to preset second gain information and transmitting a second pre-emphasis signal through a second transmission line, when the processed image signal is abnormal; and
performing the equalizing operation according to preset second correction information with respect to the second pre-emphasis signal.

10. The method of claim 9, further comprising a dual transmission process of switching from the first transmission line to the second transmission line, when the post-processing image signal is abnormal.

11. The method of claim 9, wherein the first gain information and the first correction information are used to correct signal distortion according to an impedance of the first transmission line,
wherein the second gain information and the second correction information are used to correct signal distortion according to an impedance of the second transmission line.

12. The method of claim 9, the first gain information and the second gain information are stored in a transmission lookup table, and
the first correction information and the second correction information are stored in a reception lookup table including.

13. The method of claim 9, further comprising:
transmitting a first hash value generated in a process of being compressed by a hash function through a monitoring transmission line different from the first transmission line and the second transmission line, when the pre-emphasis is performed for the image signal compressed by the hash function, and
determining whether the post-processing image signal is normal by comparing a second hash value generated according to the hash function after decompressing the post-processing image signal with the first hash value.

14. The method of claim 13, wherein a transmission speed of the monitoring transmission line is slower than a transmission speed of the first transmission line and the second transmission line.

15. The method of claim 9, wherein the first pre-emphasis signal transmitted through the first transmission line includes redundant information,
further comprising checking the redundant information from the post-processing image signal and determining whether the post-processing image signal is normal.

16. The method of claim 15, further comprising:
transmitting a switching control signal from the image signal receiving unit, when a check result of the redundant information is abnormal; and
a dual transmission process of switching from the first transmission line to the second transmission line according to the switching control signal.

17. The endoscope apparatus of claim 2, wherein the first gain information and the first correction information are used to correct signal distortion according to an impedance of the first transmission line,
wherein the second gain information and the second correction information are used to correct signal distortion according to an impedance of the second transmission line.

18. The endoscope apparatus of claim 2, wherein the image signal transmitting unit stores a transmission lookup table including the first gain information and the second gain information,
wherein the image signal receiving unit stores a reception lookup table including the first correction information and the second correction information.

19. The method of claim 10, wherein the first gain information and the first correction information are used to correct signal distortion according to an impedance of the first transmission line,
wherein the second gain information and the second correction information are used to correct signal distortion according to an impedance of the second transmission line.

20. The method of claim 10, the first gain information and the second gain information are stored in a transmission lookup table, and
the first correction information and the second correction information are stored in a reception lookup table including.
